(19) United States
(12) Patent Application Publication
(10) Pub. No.: US 2010/0305416 A1
(43) Pub. Date: Dec. 2, 2010

(54) NON-INVASIVE PRESSURED PROBING DEVICE

(75) Inventors: Michel Bédard, St-Augustin-de-Desmaures (CA); Dany Nolet, Ancienne-Lorette (CA); Nelson Landry, St-Augustin-de-Desmaures (CA)

Correspondence Address: CHOATE, HALL & STEWART LLP, TWO INTERNATIONAL PLACE, BOSTON, MA, 02110, US

(73) Assignee: CYBIOCARE, INC., Quebec (CA)

(21) Appl. No.: 12/599,102

(22) PCT Filed: Oct. 23, 2007
(86) PCT No.: PCT/CA07/01889

§ 371 Date: Nov. 6, 2009

Related U.S. Application Data
(60) Provisional application No. 60/924,277, filed on May 7, 2007.

(51) Int. Cl. A61B 5/145

(52) U.S. Cl. 600/309

(57) ABSTRACT

A non-invasive probing device wearable over a skin of a patient for monitoring the patient is provided. The probing device includes a physiological probe having a sensing interface, and a housing for housing the probe. The housing includes a body having a skin-side surface for lying next to the skin of the patient and a fixed or resilient protruding member fixedly or resiliently protruding from the skin-side surface. The protruding member houses the sensing interface. The device also includes attachment means for attaching the device to the skin of the patient. In the case of a resilient protruding member, the device may include a resilient mechanism for biasing the resilient protruding member against the body. Also in the case of a resilient protruding member, the device may include a protective cover for protecting the device against unwanted external influences.

### FIELD OF THE INVENTION

The present invention relates to the field of medical devices and more particularly concerns a non-invasive probing device, including a physiological probe having a reduced sensitivity to unwanted variations in pressure.

### BACKGROUND OF THE INVENTION

In recent years, the development of novel methods of measurement and monitoring of analyte levels in human tissues has been one of the hot topics of biomedical diagnostics. More particularly, glucose is of special interest due to an increase in the number of diabetes patients. The World Health Organization (WHO) estimates that more than 180 million people worldwide have diabetes. This number is likely to more than double by 2030. Non-invasive methods are the most promising because they potentially allow to avoid frequent finger-pricking blood sampling and to provide continuous monitoring of the glucose levels in the blood or interstitial fluid (ISF).

Non-invasive optical technologies have great potential in biology, medicine and sports because they have the potential to provide real time information to the user or the medical personnel by tracking various physiological parameters or states. The recent proliferation of optical communication systems and the availability of such products have driven the industry to produce low-cost and reliable optical sources and detectors, making their use in optical glucose measurement systems particularly attractive. Although many studies have shown this great potential, very few investigators have been able to completely isolate the signal of interest from the various interferences that come from the external environment and obtain precise signals that can be correlated with glucose levels. This is due in part to the fact that the measurements must be made in a continuous manner on a constantly moving subject and to the variable nature of the human body itself. Also, the elastic nature of human tissue complicates the taking of optical measurements when a subject is in motion since tissue compression and expansion instantly affect the optical properties of the tissue while the signal of interest remains fairly constant. In general, these constraints also apply to other non-invasive probes, for example bio-impedance analyzers.

International patent application published under No. WO 01/067946 discloses a probe device for use in non-invasive optical measurements of a least one parameter of the blood of a user, and relates to a probe to be uniquely applied to a finger of a patient. In this case, the main problem with this approach is that a substantially under-systolic pressure needs to be applied to the measurement location and, in order to obtain accurate results, at least two timely separated measurement sessions should be considered. Furthermore, since the device is placed on the fingertip, it is conspicuous and can be influenced by many external disturbances.

International patent application published under No. WO 05/077260 discloses a procedure and apparatus for determining a physiological parameter such as heart rate, blood pressure, and blood chemistry analytes including glucose, lactate and oxygen saturation. When used to monitor a specific parameter, e.g. glucose, this invention is limited by the accuracy of the more accurate of the glucose sensors used. Moreover this document does not provide any solution regarding the effects of external force or instability on the device.

U.S. Pat. No. 6,402,690 discloses a monitoring system for monitoring the vital signs of a patient by performing measurements such as skin temperature, blood flow, blood constituent concentration, and pulse rate at the finger of a patient. Physically the monitoring system has an inner ring close to the surface of the skin, as well as an outer ring mechanically decoupled from the inner ring thereby shielding it from external loads. The main problem with this approach is that it is limited to use on a finger, and is therefore especially subject to movements in real-life usage and not convenient since a device on a finger represents a handicap for the user.

New non-invasive approaches for measuring the level of blood glucose have been pursued. For example, U.S. Pat. No. 7,139,076 discloses an apparatus and methods for stable and reproducible optical diffuse reflection measurements. A 2×2 optical probe with light emitting diodes (LEDs) as illumination sources and photodetectors as detection means are used to make the measurements. The surface instabilities are presumably cancelled out and accuracy is presumably increased since only deeper layers of the sample would contribute to the result. The main problem with this approach is that it does not reduce motion artifacts and specifically does not reduce the sensitivity to external pressure or force of the device.

According to literature (Heinemann et al., “Non-invasive glucose measurement by monitoring of scattering coefficient during oral glucose tolerance tests”, Diabetes Technology & Therapeutics, 2(2): 211-220, 2000), small movements of the sensor head of non-invasive probes due to imperfect fixation or external forces, may lead to considerable signal drifts. Therefore, all the more reason to isolate these motion artifacts and reduce their impact on the measured signal. Moreover, previous studies have demonstrated that tissue optical properties are changed under compression. In other words, it is important to resolve the pressure effects on soft tissues (Shangguan et al., “Pressure effects on soft tissues monitored by changes in tissue optical properties”, Laser Tissue Interaction IX, Proc. SPIE 3254: 366-371 1998; and Chan et al., “Effects of compression on soft tissue optical properties”, IEEE J. selected Topics in Quantum Electronics 2: 943-950, 1996).

One of the most difficult problems in implementing a probing device that is wearable on the body is the issue of eliminating or nevertheless reducing signal artifacts due to motion of, or forces exerted upon, the sensor. The pressure due to the contact probe may also affect, for example, the optical properties derived because of altered local blood content.

As such, the present invention aims to alleviate at least some of the drawbacks of the prior art.

### SUMMARY OF THE INVENTION

In accordance with a first aspect of the invention, there is provided a non-invasive probing device wearable over a skin region of a patient for physiological monitoring thereof. The probing device includes: a physiological probe having an sensing interface; a housing for housing the probe, the housing including a body having a skin-side surface for lying adjacent to the skin region of the patient, the housing further including a fixed protruding member protruding from the skin-side surface away from the body, and toward the skin region of the patient, the protruding member housing the sensing interface; and attachment means for attaching the housing to the skin region of the patient, and holding the skin-side surface and protruding member in pressure contact over the skin region.

The sensing interface may include at least one output of a light guide. It may include at least one input of a light guide.

The probing device may further include a rigid protective cover enclosing the housing, the cover having an outlet therein for the protruding member.

The attachment means may include an adhesive layer extending over the skin-side surface of the body. In accordance with one embodiment, the adhesive layer surrounds the protruding member and has a thickness less than a length of the protruding member.

Alternatively, or in addition, the attachment means may include a flexible band or strap for encircling a body part of the patient and holding the housing of the probe in pressure contact over the skin region of the patient. In accordance with one embodiment, the strap or band is elastic.

Alternatively, or in addition, the attachment means may include medical-grade adhesive tape for taping over the housing and securing the housing to the skin region of the patient, the medical-grade adhesive tape including adhesive strips for adhering to the housing and the skin region of the patient and thereby securing the housing to the skin region of the patient.

Alternatively yet, the attachment means may include a medical-grade adhesive patch having a non-adhesive portion for covering the housing and an adhesive portion for adhering to the skin region of the patient, thereby securing said housing to the skin region of the patient.

In accordance with a second aspect of the invention, there is provided a non-invasive probing device wearable over a skin region of a patient for physiological monitoring thereof, which includes: a physiological optical probe having a sensing interface; a housing for housing the probe, the housing including a body having a skin-side surface for lying adjacent to the skin region of the patient, the housing further including a resilient protruding member resiliently protruding from the skin-side surface away from the body and toward the skin region of the patient, the resilient protruding member housing the sensing interface; and attachment means for attaching the housing to the skin region of the patient and holding the skin side surface and protruding member in pressure contact over the skin region.

The sensing interface may include at least one output of a light guide. It may include at least one input of a light guide.

The resilient protruding member may be retractable within the body. Alternatively or additionally, it may be extendable without the body.

The non-invasive probing device may further include a rigid protective cover enclosing the housing and having an outlet therein for the protruding member.

The non-invasive probing device may include a resilient mechanism for resiliently biasing the resilient protruding member against the body. In accordance with one embodiment, the resilient mechanism may include an elastic band for resiliently biasing the body against the protective cover. In accordance with another embodiment, the resilient mechanism may include a spring for resiliently biasing the resilient protruding member against a surface opposite the skin-side surface of the body.

The non-invasive optical probing device may further include a limit sensor operably connected between the spring and the body of the housing.

The attachment means may include any of the elements described earlier hereinabove.

The objects, advantages and other features of the present invention will become more apparent and be better understood upon reading of the following non-restrictive description of the preferred embodiments of the invention, given with reference to the accompanying drawings. The accompanying drawings are given purely for illustrative purposes and should not in any way be interpreted as limiting the scope of the invention.

### BRIEF DESCRIPTION OF THE DRAWINGS

FIGS. 1A and 1B (PRIOR ART) are respectively a bottom view and a cross-sectional of a simple physiological optical probe; FIG. 1C (PRIOR ART) is a block diagram of the physiological optical probe of FIGS. 1A and 1B.

FIG. 2 is a cross-sectional view of a non-invasive probing device according to one embodiment of the invention, showing a fixed protruding member.

FIG. 3A is a graph of the measured signal variation as a function of the pressure applied on a non-invasive probing device without a protruding member; FIG. 3B is a graph of the measured signal variation as a function of the pressure applied on a non-invasive probing device with a fixed protruding member as shown in FIG. 2.

FIG. 4 is a cross-sectional view of a non-invasive probing device according to another embodiment of the invention, showing a resilient protruding member.

FIG. 5 is a schematic representation of a force diagram for a non-invasive probing device with a resilient protruding member.

FIG. 6 is a graph of the modelized skin to sensor force (F₂) as a function of the external pressure (Fₑ) applied on device of FIG. 5, for different spring constants.

FIG. 7 is a graph of the device height (d₁), probe height (d₂) and probe to device height (d₂−d₁) as a function of the external pressure (Fₑ) applied on the device of FIG. 5.

FIG. 8 is a schematic cross-sectional view of a non-invasive probing device with a resilient springy body, according to yet another embodiment.

FIG. 9 is a schematic diagram of a patient wearing a non-invasive probing device attached by a band or strap according to an embodiment of the invention.

FIG. 10 is a schematic diagram of a patient wearing a non-invasive probing device attached by an adhesive patch according to another embodiment.

FIG. 11A is a schematic side-view diagram of a non-invasive probing device, showing attachment means according to an embodiment; FIG. 11B is a schematic top-view diagram of FIG. 11A.

### DETAILED DESCRIPTION OF PREFERRED EMBODIMENTS OF THE INVENTION

The present invention relates to non-invasive probing devices wearable over a skin region of a patient for the physiological monitoring of this patient. The term “wearable” is understood herein to refer to the action of keeping a device according to the invention in close proximity to the skin of the patient for a predetermined period of time sufficient to accomplish the desired physiological monitoring. The skin region of the patient can be on any appropriate location of the body of the patient (e.g. abdomen, side of abdomen, arm etc.), and is understood to include the precise location probed by the device, as well as the surrounding area.

Various examples of non-invasive probing devices according to preferred embodiments of the present invention are illustrated in the accompanying drawings. Each probing device generally includes a physiological probe having a sensing interface, a housing for housing the physiological optical probe and means for attaching the housing to a skin region of the patient. The housing of the non-invasive probing device includes a body that has a skin-side surface, this surface being generally flat for lying against the skin of the patient, and a protruding member protruding from the skin-side surface away from the body of the housing. It should be noted that the expressions “skin-side surface” is used to denote a surface that faces the skin of the patient as opposed to a surface that faces externally, away from the patient. It should in no way be used to limit the surface to one that is in contact with the skin of the patient, albeit that such a case is possible.

In the following description, a diffuse-reflectance physiological optical probe will be described as an embodiment of the physiological probe. Of course, as any one skilled in the art will understand, this is done for the sake of clarity and conciseness and should in no way be interpreted as limiting the invention to such a case. The non-invasive probing device may include a physiological probe, with an appropriately adapted sensing interface, based on, for example, spectral analysis, optical spectroscopy, diffuse-reflectance measurements and bio-impedance analysis.

### Description of an Exemplary Physiological Probe

The diffuse-reflectance physiological probe may for example be of any type known in the art. Such a probe may be used to measure physiological characteristics typically by collecting the backscattered light of the illuminated tissue site, i.e. the diffuse reflectance. In order to obtain the optical response of the tissue, a source illuminates a spot on the skin surface and one or more light sensors monitor reflected light around this spot. The final resultant signal of diffuse reflectance is correlated with the analyte, e.g. glucose, content in the interstitial fluid, i.e. with the light beam signal from the interstitial fluid (ISF). It is well documented in literature that the optical properties of the ISF change under the soaking influence of glucose. In fact, the variation of analyte levels, in this example blood glucose levels, changes the light scattering properties of skin tissue and consequently affects the measured reflectance profile.

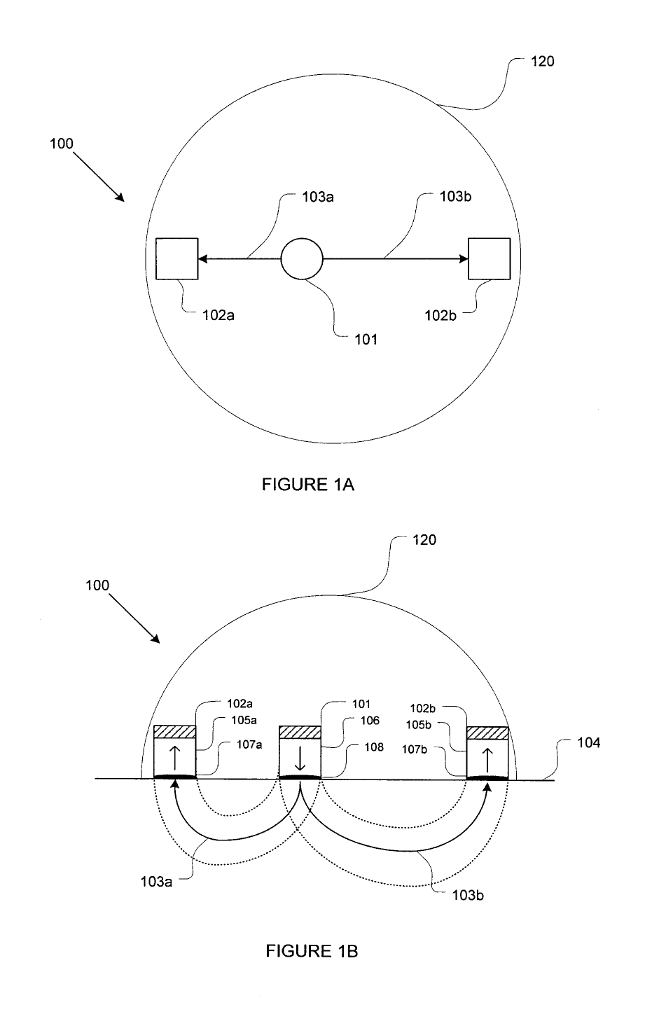

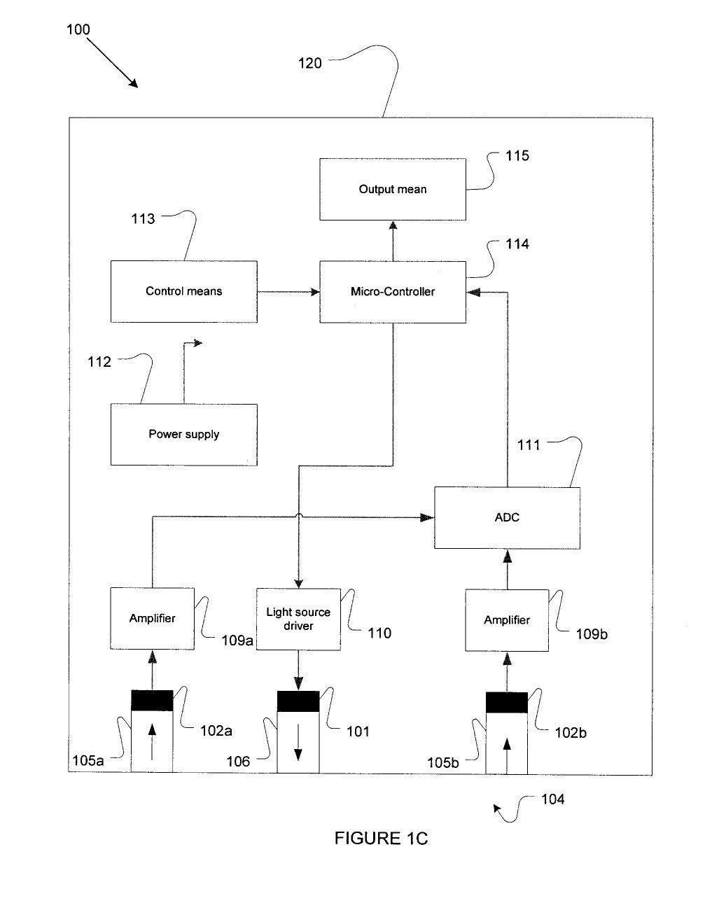

Referring to FIGS. 1A to 1C, (PRIOR ART), the typical components of a physiological optical probe of this type are illustrated. FIG. 1A (PRIOR ART), shows a bottom view, as would be viewed from the skin surface, of a typical basic optical probing device 100 incorporating such a probe. It is understood that the designations “bottom”, “top” and “side” are used herein to describe the probe for convenience of reference only and are not meant to denote any preferential orientation of the probing device. One light source 101 and two light detectors 102a and 102b are shown on the bottom surface of the optical probing device 100. Each arrow corresponds to directions of the light beams 103a and 103b and each respectively corresponds to different distances between the light source and the detectors. This figure is used to demonstrate an optical probe in its simplest form and to show how diffuse reflectance measurements are obtained with this device. The housing 120 of the basic typical optical probing device 100 in this example is circular in shape, however it is not limited to this shape and the light source-detector configuration is also not limited to this pattern.

FIG. 1B (PRIOR ART) is a schematic side view of the same optical probing device 100. A typical light beam is emitted from light source 101. It then passes through a light guide 106, exits into the skin through the output 108 of the light guide 106, travels through the skin by way of 103a or 103b, enters the probe through inputs 107a or 107b of light guides 105a and 105b respectively, at which point the light beam passes through the light guides 105a or 105b and is finally captured by light detectors 102a or 102b.

FIG. 1C (PRIOR ART) schematically illustrates the inner components of the physiological optical probe housed in the housing 120 of the optical probing device 100 of the preceding figures. All the components that need to be powered are shown to be fed by power supply 112. Once the physiological optical probe is functional, the micro-controller 114 feeds the light source driver 110 which then provides current to light source 101. The light source 101 emits light beams through a light guide 106 and then the probing light beams propagate into the dermis of the skin 104. Light beams scattered back from the dermis of the skin 104 preferably pass through other light guides 105a and/or 105b, and are then received by the light detectors 102a and/or 102b.

An Analog to Digital Converter (ADC) 111 converts the analog signals from the light detectors 102a and 102b, as amplified by their respective amplifiers 109a and 109b, into digital signals which are fed to a micro-controller 114. The operations of the optical physiological probe may be controlled by control means 113, for example a processor. The micro-controller is also connected to an output device 115 through which the resulting data or measurements are provided to the user. The data is analyzed by micro-controller 114 in order to obtain measurements which may be correlated with analyte levels, e.g. glucose levels. To evaluate the different light beams, the intensity of each signal I₁₀₂ₐ and I_{102b} is measured and then the ratio R gives the diffuse-reflectance measurement (see Equation 1), known to be correlated with glucose.

$$R = \frac{{I}_{102 a}}{{I}_{102 b}} \qquad Equation 1$$

As mentioned above, the physiological probe may be based on any other appropriate non-invasive technology allowing the monitoring of a physiological characteristic such as glucose, or a clinical state (for example hypoglycemia), and detects or predicts if the patient under monitor is affected by such a state. In order to measure such a physiological characteristic or to detect such clinical states, the physiological probe should be as stable as possible, hence insensitive to motion artifacts and pressure, and should be able to give accurate in vivo measurements, either absolute or relative.

### Probing Device According to a First Embodiment

Physiological optical probes are placed in pressure contact with the tissue under monitor so as to reduce index mismatch and increase light transmittance and, as mentioned above, the optical tissue responses of such probes however can be very sensitive to the application of pressure. The pressure applied to a probing device worn by a patient can vary greatly both from patient to patient and over time for a given patient, and for example depend on the manner in which the device is attached to the patient, the location of the device on the patient's body, the type of clothing worn over the device, external forces accidentally applied on the device by the patient or his surroundings, etc. It has been found that the detrimental effects of these pressure variations can be greatly alleviated by providing the probe within a housing having a protruding member (i.e. part), either fixed or variable, that applies either a fixed minimum or a controlled pressure on the tissue (typically the skin) of the patient under monitor to stabilize the tissue response and consequently the physiological measurements made by the probe.

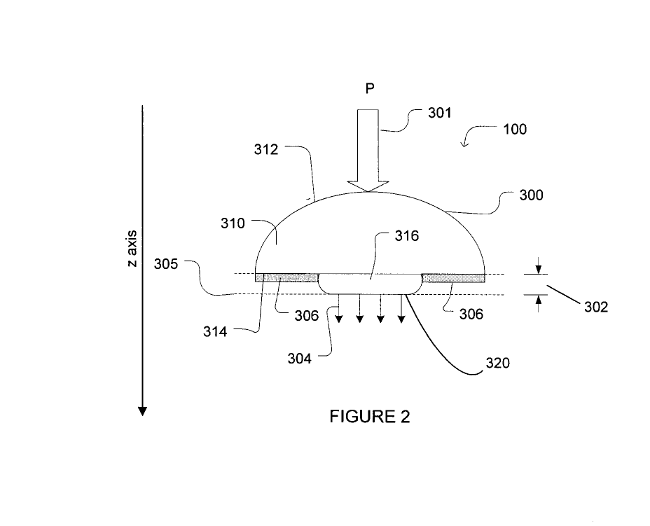

FIG. 2 shows in cross-sectional view a non-invasive probing device 100 including a physiological probe provided in a housing 300 according to a first preferred embodiment of the present invention.

This embodiment can accommodate a wide variety of physiological probe types, including optical diffuse-reflectance probes such as the one previously described in FIG. 1A to 1C. The physiological probe may be any appropriate probe for non-invasive monitoring of a patient through the skin and may use, for example, spectral analysis, optical spectroscopy, diffuse-reflectance measurements and bio-impedance analysis to monitor the patient. The physiological probe has a sensing interface, which may be embodied by any component of the probe from which sensing signals are sent and received. In the case where the physiological probe is optical in nature, the optical sensing interface 320 may include one or many light guides. The light guide may be any appropriate optical waveguide, for example an optical fiber. The optical sensing interface preferably includes at least one output of a light guide for interrogating the skin tissue site under monitoring and one input of a light guide for receiving a response signal from the interrogated site. In addition, the physiological probe may include an appropriate detector for detecting the response received from the interrogated site via the input of the light guide. Moreover, the physiological probe may include a processor capable of processing the clinical information in the detected response signal from the interrogated site.

The housing 300 includes a body 310 having a top surface 312 and a bottom skin-side surface 314. The skin-side surface 314 is generally flat for lying against the skin of the patient. In the illustrated embodiment, the top surface of the body is shown to be dome-shaped, but one skilled in the art will understand that it could have a different contour without departing from the scope of the present invention. The housing 300 further includes a protruding member 316 protruding from the bottom skin-side surface 314 of the body 310 away from the body. As shown, the protruding member 316 is preferably centrally located on the skin-side surface of the body. Of course, the protruding member may be located anywhere along the skin-side surface that does not interfere with the measurements.

The protruding member 316 houses the sensing interface 320. The other components of the physiological probe are housed either in the protruding member 316 or the body 310 of the housing 300. For example, in the case of an optical probing device, the protruding member may house the other optical components of the physiological probe, that is, the light sources, light guides, detectors, etc, as explained above, in addition to the optical sensing interface while the body may house the other components of the probe such as the power supply, micro-controller, etc. Of course, any appropriate arrangement of the components of the physiological optical probe within the housing is possible. For example, in the case of an optical probing device, the electronic components (e.g. processor, etc) as well as the majority of the optical components (light guides, detector, etc) may be housed within the body 310 of the housing 300 while the protruding member 316 houses the optical sensing interface 320.

Nevertheless, the protruding member 316 houses the sensing interface 320. The sensing interface 320 is preferably located at the end of the protruding member 316. It is the sensing interface that is placed in contact with the skin of the patient when physiologically monitoring the patient. Preferably, the end of the protruding member is relatively smooth with a rounded profile. Of course, the shape of the end of the protruding member may be any appropriate shape that does not cause injury to the site under monitor.

In the embodiment of FIG. 2, the protruding member 316 is fixed with respect to the body 310 of the housing. The distance between the sensing interface of the protruding member and the skin-side surface of the body of the housing is such that in use, the protruding member applies a constant fixed minimum pressure on the skin of the patient, thereby advantageously reducing the sensitivity of the physiological probe to unwanted extraneous vibration, external motion artifacts or pressure 301. The application of pressure 304 on the skin of the patient stabilizes the response of the adjacent tissue. It has been found that a distance (302) of the order of 1 to 7 mm separating the normal skin level 305, from the bottom of the protruding optical physiological probe 100 can be sufficient, in some embodiment, to attain the objectives of the present invention. Of course, this distance can vary greatly without departing from the scope of the present invention.

The probing device 100 further includes attachment means for attaching the housing to the skin region of the patient. In the embodiment of FIG. 2, the attachment means are embodied by an adhesive layer 306, which extends over the skin-side surface 314 of the body 310 and surrounds the protruding member 316, for attaching the housing to the skin of the patient. The adhesive layer 306 has a thickness adapted so that the minimum pressure on the skin of the patient is provided. For example, the adhesive layer may surround the protruding member and may have a thickness less than a length of the protruding member, i.e. less than the distance from the skin-side surface to the optical sensing interface end 320 of the protruding member 316. The adhesive layer pulls the skin around the protruding member allowing the protruding member to press against the skin, and thereby create the pressure mentioned above. Other attachment means may be provided alternatively or additionally to the adhesive layer, as will be explained in more detail further below.

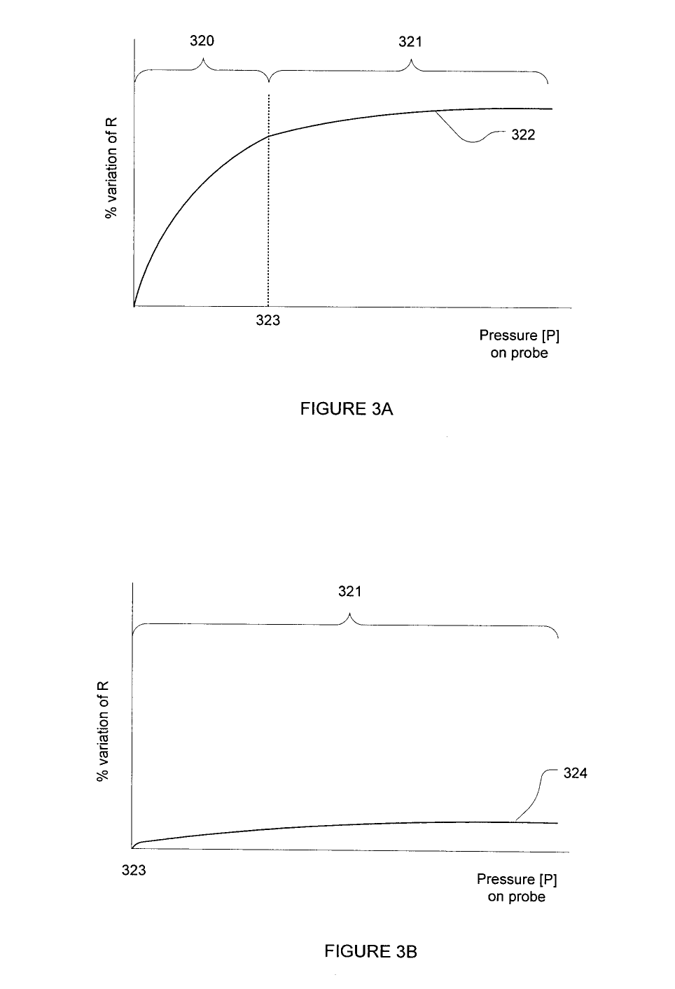

Referring to FIG. 3A, there is shown a plot of the absolute value of the measured signal variation (R from Equation 1) as a function of the pressure (analogous to pressure 301 in FIG. 2) applied on an optical probing device, as it would be if the housing was not provided with the protruding member 316 shown in FIG. 2. In this example, there are two distinct regions: a first region 320 where the signal variation is considerable compared to the amount of external pressure applied on the probing device. In a second region 321, after a minimum amount of pressure is reached 323, the variation of signal 322 is not as significant. However, when using an optical probing device with a fixed protruding member, only region 321 is effective. The protruding member applies a constant pressure on the skin of the patient and therefore the physiological probe is much less sensitive to external disturbances, as can be seen in FIG. 3B. When the amount of pressure applied on the optical probing device increases, the measured signal barely varies as is illustrated from curve 324.

### Probing Device According to a Second Embodiment

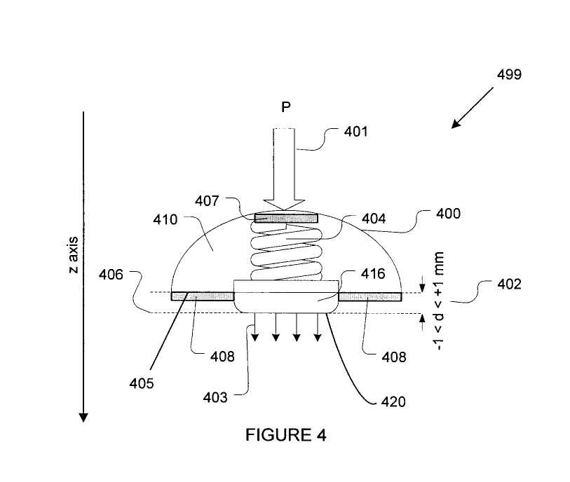

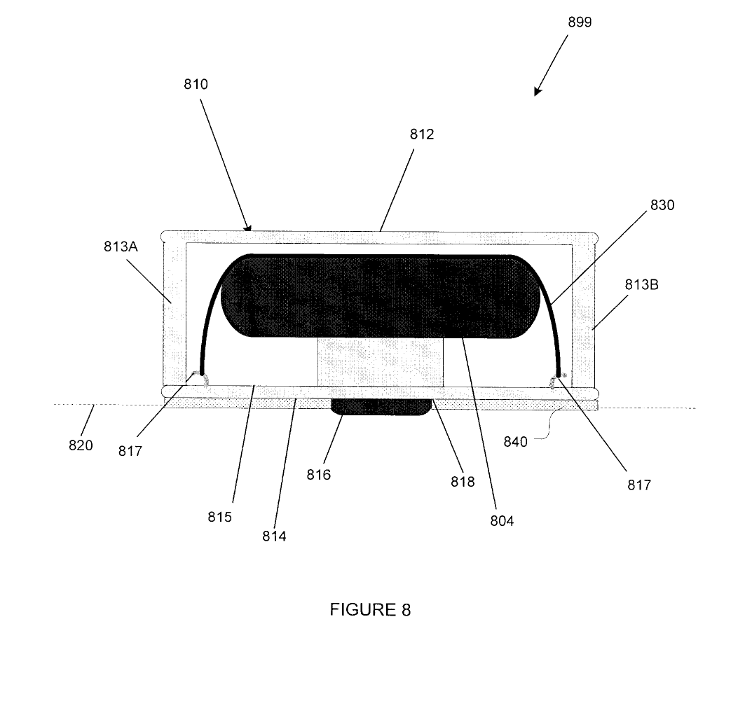

An alternative embodiment of a probing device according to the present invention is illustrated in FIG. 4. This embodiment may be preferred for physiological probes based on optical spectroscopy such as those demonstrated in the following: U.S. Pat. No. 7,133,710, U.S. Pat. No. 7,039,447, U.S. Pat. No. 6,865,408. In this embodiment, the housing 400 of the probing device 499 also includes a body 410, a protruding member 416, housing a sensing interface 420, and an adhesive layer 408, with the difference that the protruding member 416 is resiliently biased within the body 410. The housing may further include a resilient mechanism for resiliently biasing the resilient protruding member against the body of the housing. Here the housing is dome-shaped, but of course the housing may have any appropriate shape. In the illustrated embodiment of FIG. 4, a spring 404 is shown biasing the resilient protruding member 416 against the body 410 of the housing 400, but other resilient mechanisms (e.g. an elastic band) could be used as shown in FIG. 8 described further below. The resilient protruding member is resiliently biased against the body of the housing and is both retractable within the body of the housing and extendable without. Moreover, the resilient protruding member applies a constant minimum pressure on the skin of the patient. For appropriate low spring/elastic constants of the spring or elastic of the resilient mechanism, the resilient protruding member may retract fully into the body, applying a negative pressure—the force of the skin of the patient is greater than the force of the resilient protruding member. Owing to the resilient nature of the resilient protruding member, the physiological probe has a reduced sensitivity to vibrations and unwanted external motion artifacts or forces.

Since this device is spring-controlled, the amount of pressure applied on the skin of the patient by way of an external force is greatly reduced. For use in spectroscopy, this is particularly convenient since the probing light beam must propagate in the same skin layers time after time in order to accurately analyze the content of the chemical analytes under study in the biological sample. If excessive pressure is applied on the tissue, the skin layers will be compacted and instead of measuring analytes solely in the desired skin layers, other deeper layers of tissues will be seen by the physiological probe. In this embodiment, the spring 404 is mounted inside the body 410 of the housing 400 in order to apply a variable controlled force 403 and transmit it to the adjacent tissue depending on the skin characteristics of the patient and spring constant. When an external pressure 401 is applied to the housing 400, the spring contracts, the distance 402 between the skin-side surface 405 and the normal skin level 406 is reduced reducing the force applied to the skin. Furthermore, a limit sensor 407 may be added to the top of the spring to warn when too much pressure is applied and the spring can no longer compensate, and hence prevent the physiological probe measurements from being erroneous or inexact.

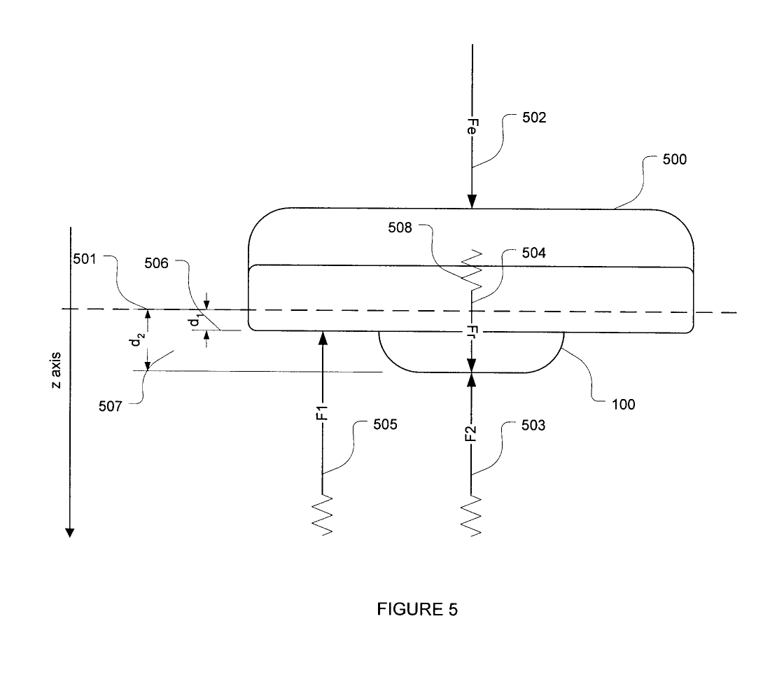

Reduction of external forces is best demonstrated with simulations conducted using a theoretical mechanical model as seen in FIG. 5. All the values indicated for forces (F₁, F₂, Fₑ, Fᵣ) and distances (d₀, d₁, and d₂) are for illustration purposes only and may be different for a given application. From the schematic diagram of FIG. 5, the following equations can be derived:

F₁=k₁·d₁ Equation 2

F₂=k₂·d₂ Equation 3

where
F₁ is the force 505 of the skin on the sitting contour, i.e. the skin-side surface of the body of the housing, of the probing device 500
F₂ is the force 503 of the skin on the protruding member of the physiological probe 100
d₁ is the distance 506 between the normal skin level 501 (z=0) and the surface of the sitting contour, i.e. the skin-side surface of the body of the housing, of the probing device 500
d₂ is the distance 507 between the normal skin level 501 (z=0) and the surface of the protruding member of the physiological probe 100
k₁, k₂ are spring constants presented from the skin under the skin-side surface of the body, i.e. sitting contour, of the probing device 500 and physiological probe 100 respectively.

We may further derive the following equations:

Fₑ=F₁+F₂ Equation 4

Fᵣ=kᵣ·[d₀(d₂−d₁)]; where d₀=1 mm Equation 5

where
Fₑ is the external force 502 applied on the probing device 500
Fᵣ is the force 504 produced by the spring 508 on top of the physiological probe 100
kᵣ is the constant of the spring 508 on top of the physiological probe 100
d₀ is defined as the initial protrusion when F₁ and F₂ are null

Using Equations 2, 3 and 4, we obtain:

Fₑ=k₁·d₁+k₂·d₂ Equation 6

We may then isolate d₁:

$${d}_{1} = \frac{{F}_{e} - {k}_{2} \cdot {d}_{2}}{{k}_{1}} \qquad Equation 7$$

Also, knowing opposite forces need to be equal in order to obtain equilibrium, we obtain:

Fᵣ=F₂ Equation 8

Using Equations 5 and 8, we obtain:

k₂·d₂=kᵣ·[d₀−(d₂−d₁)] Equation 9

We may then isolate d₂:

$${d}_{2} = {k}_{r} \cdot \frac{{d}_{0} + {d}_{1}}{{k}_{2} + {k}_{r}} \qquad Equation 10$$

Using Equations 7 and 10, we may substitute d₂ by its equivalent and replace it in Equation 7 to obtain:

$${d}_{1} = \frac{[ {F}_{e} - {k}_{2} \cdot ( {k}_{r} \cdot \frac{{d}_{0} + {d}_{1}}{{k}_{2} + {k}_{r}} ) ]}{{k}_{1}} \qquad Equation 11$$

Equation 11 may be rewritten as follows:

$${d}_{1} = \frac{( {F}_{e} \cdot {k}_{2} + {F}_{e} \cdot {k}_{r} - {k}_{2} \cdot {k}_{r} \cdot {d}_{0} )}{{k}_{1} \cdot {k}_{2} + {k}_{1} \cdot {k}_{r} + {k}_{2} \cdot {k}_{r}} \qquad Equation 12$$

Then using Equation 10 and 12, we may substitute d₁ by its equivalent and replace it in Equation 10 to obtain:

$${d}_{2} = {k}_{r} \cdot \frac{{d}_{0} + \frac{( {F}_{e} \cdot {k}_{2} + {F}_{e} \cdot {k}_{r} - {k}_{2} \cdot {k}_{r} \cdot {d}_{0} )}{{k}_{1} \cdot {k}_{2} + {k}_{1} \cdot {k}_{r} + {k}_{2} \cdot {k}_{r}}}{{k}_{2} + {k}_{r}} \qquad Equation 13$$

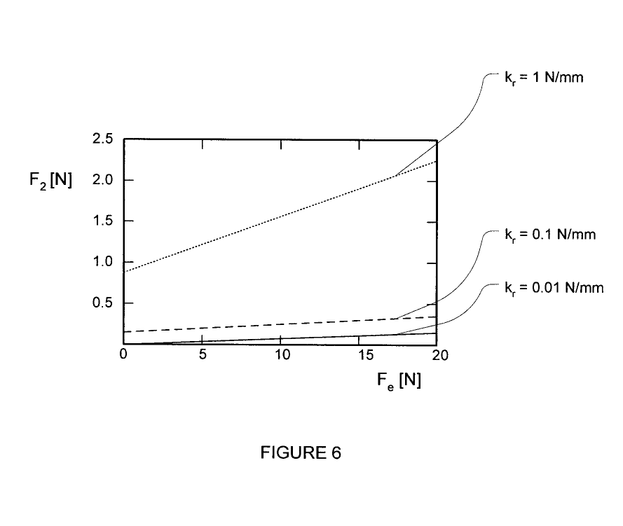

In this simulation, FIG. 6 shows F₂ plotted as a function of external force Fₑ for three fixed values of kᵣ. As can be observed, F₂ is barely affected by the increase in external pressure Fₑ when a proper small spring constant is chosen. For a higher value of the spring constant, F₂ displays more opposition to the external force and the protruding section is significantly pressed into the user's skin.

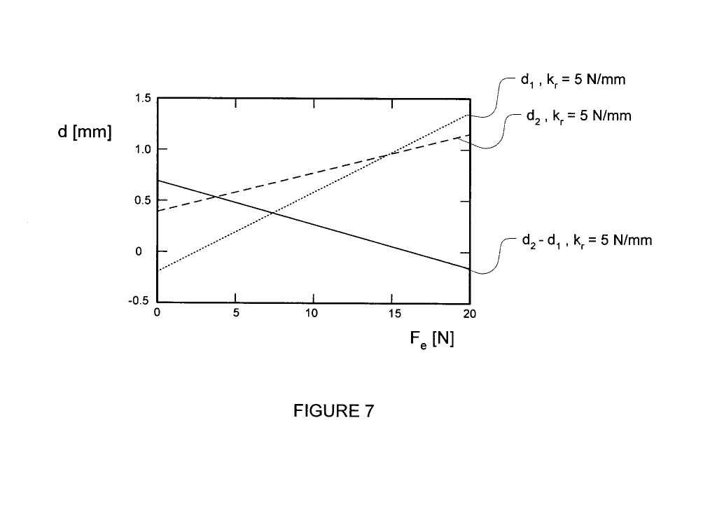

Referring to FIG. 7, d₁, d₂ and d₂−d₁ were also plotted as a function of the external force Fₑ. A value of kᵣ=5 N/mm in Equation 12 and 13 was used to visualize how these distances varied for increasing values of an external force up to a maximum of 20 N. It can be noted that as more pressure is applied on the device by an external body, d₁ increases accordingly. As Fₑ increases, more force is applied, hence the sitting contour of the device presses deeper into the user's skin, therefore d₁ increases.

Furthermore, the same analysis can be made concerning d₂. However, since d₂ already has an initial protruding section, the external force, even though it increases, barely affects the distance between the surface of the protruding section and the normal skin level. Lastly, when subtracting d₁ from d₂ to visualize how an external force affects the spring controlled protruding section, it can be noted that the protruding section has a tendency to move upwards inside the housing (d₂−d₁ being negative). This phenomenon can be explained by the fact that since the spring constant is quite low, when an external pressure is applied on the outer shell, the sitting contour transfers all the pressure to the surface of the skin. Thus, creating a region where the protruding section is acting in the opposite direction of Fᵣ.

### Probing Device According to a Third Embodiment

Referring to FIG. 8, there is shown a schematic cross-sectional view of another embodiment of a non-invasive probing device 899. This embodiment has a rigid protective cover in addition to the housing.

In order to reduce or eliminate the detrimental effects of external vibrations, shocks and forces on the measurements taken by the non-invasive probing device, it may be desirable to insulate the probing device from these unwanted external vibrations, shocks. Advantageously, the protective cover serves to insulate the probing device so as to reduce its sensitivity to these unwanted influences.

The rigid protective cover 810 encloses the body 804 of the physiological probe. It has a top surface 812, lateral walls 813A and 813B, a bottom skin-side surface 814 that is generally flat for lying against the skin 820 of the patient, and an outlet 818 in the bottom surface 814 for allowing the resilient protruding member 816 to protrude therefrom. Preferably, the outlet 818 for the resilient protruding member 816 is centrally located in the skin-side surface 814 of the rigid protective cover. Although the protective cover 810 depicted has a flat top surface 812 and lateral walls 813 A and 813B, the top surface and lateral walls may be curved, dome-shaped, or any other shape. Furthermore, the general relative dimensions of the protective cover may differ for different embodiments.

A resilient mechanism is used to bias the body of the physiological probe 804 against the protective cover 810. In the illustrated embodiment, the resilient mechanism includes an elastic band 830 stretched over the physiological probe 804 and attached to the internal side 815 of the bottom surface 814 of the body 810 via hooks 817. The resilient mechanism serves to stabilize the pressure exerted by the protruding member 816 onto the skin and thereby stabilize the measurements of the probing device 899.

An adhesive layer 840 on the bottom surface 814 of the protective cover is used to attach the device to the skin of the patient.

### Attachment Means

In general, the non-invasive probing device includes attachment means for attaching the optical probing device to the skin of the patient. The attachment means allow the device to be placed in pressure contact with the skin of the patient. Moreover, the attachment means preferably allow for the optical probing device to be worn by the patient for an extended period of time thus allowing for long-term monitoring of a physiological condition.

As previously mentioned, the attachment means may include an adhesive layer which extends over the skin-side surface of the body (306 in FIG. 2, 408 in FIG. 4, and 840 in FIG. 8).

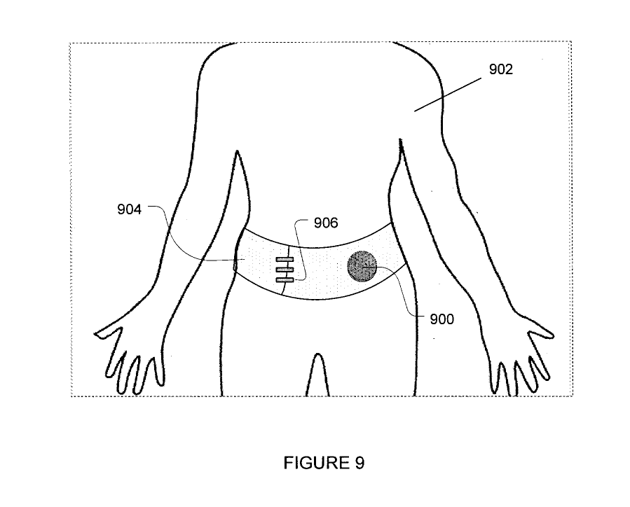

Of course, other attachment means may be used instead or in addition to the adhesive layer, examples of which are shown in FIGS. 9, 10 and 11A and 11B.

FIG. 9 is a schematic diagram showing a flexible band or strap, preferably elastic, used to encircle a body part of the patient and hold the housing in pressure contact over the skin of the patient. The non-invasive optical probing device 900 (shown in phantom line), with a fixed or resilient protruding member, is placed on a tissue of the patient 902, in this case on the skin of the patient at the waist of the patient. A flexible band or strap 904 is placed over the non-invasive optical probing device 900 and is made to encircle the waist of the patient 902. The ends of the flexible band or strap 904 are fastened together using fasteners 906, e.g. clips, safety pins or Velcro™ strips. Alternatively, for example, a flexible belt with a clasp or a buckle, or an elastic annular band may serve as the attachment means.

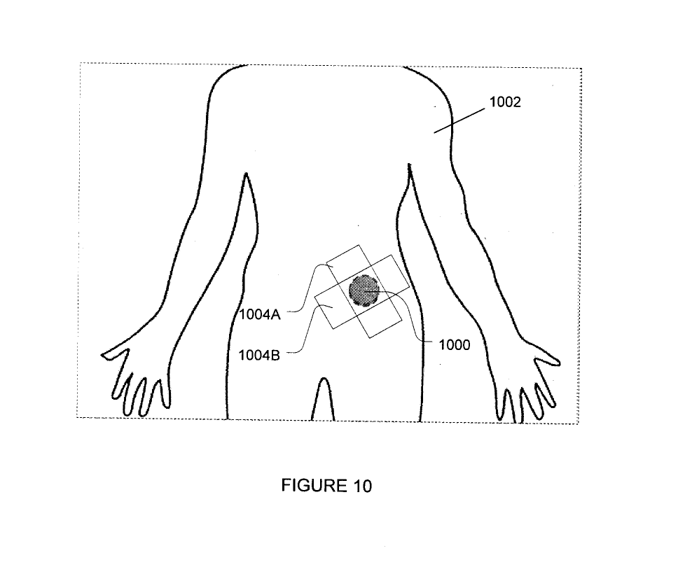

Referring to FIG. 10, there is shown medical-grade adhesive tape for taping over the housing of the non-invasive optical probing device 1000 (shown in phantom line) and securing the device to the skin of the patient. In the particular example shown, a first strip 1004A of medical-grade adhesive tape is placed over the device, adhering to the housing of the device and to the skin of the patient. A second strip 1004B is placed in crisscross fashion over the first strip 1004A to more firmly secure the device in place. It should be obvious that although there is shown two strips of adhesive tape placed in crisscross fashion, one or more strips of medical-grade adhesive tape may be placed in any pattern (parallel, crisscross, random, etc) over the device providing that the device is operationally secured in place.

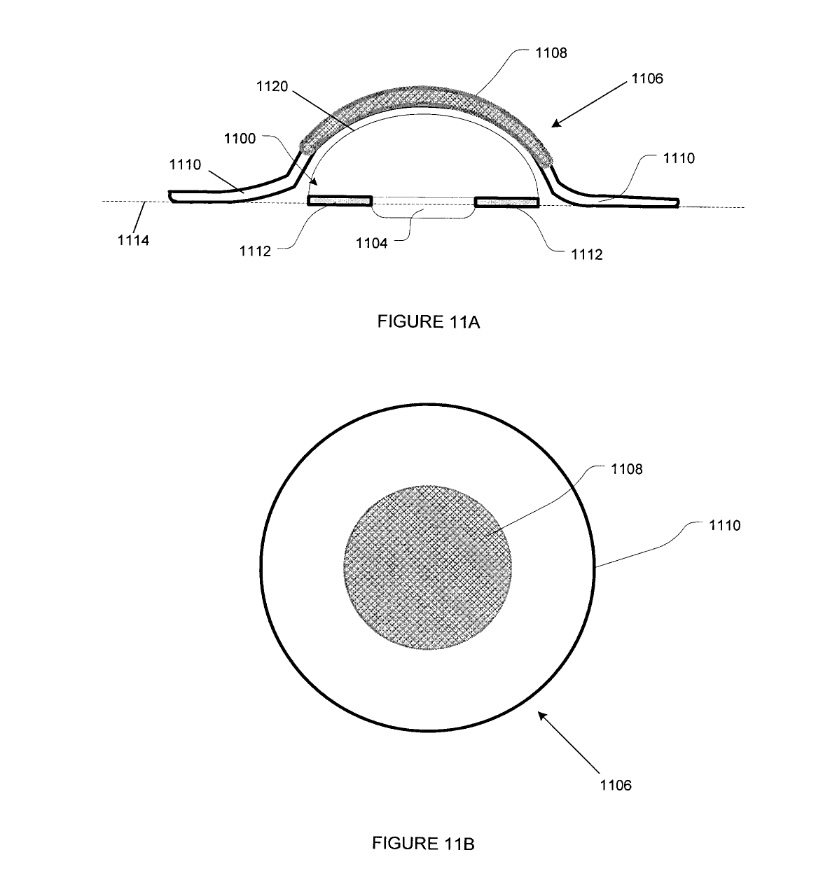

Referring to FIGS. 11A and 11B, there is given a schematic side-view and top view diagram of a non-invasive optical probing device 1100 secured to the skin 1114 of a patient using a medical-grade adhesive patch 1106 and an adhesive layer 1112 that surrounds the fixed or resilient protruding member 1104 and that is adherent to the skin 1114 of the patient. The medical-grade adhesive patch 1106 has a non-adhesive portion 1108 for covering the housing 1120 and an adhesive portion 1110 for adhering to the skin region of the patient. The non-adhesive portion 1108 may be made out of nylon or any material which allows the top of the device to slide laterally beneath the non-adhesive portion of the tape while still maintaining a pressure on the device 1100. As best seen in FIG. 11B, the adhesive portion surrounds the device and is adherent to the skin of the patient. Of course, the patch need not be circular in form, but may be any form appropriate to the form of the optical probing device.

Of course, it should be understood that numerous attachments means are possible and that the attachment means described above may be used separately or in any appropriate combination to secure the device to the patient.

In summary and advantageously, non-invasive optical probing devices with a fixed or resilient protruding member according to embodiments of the present invention allow to reduce sensitivity to unwanted external forces and to emit the light signal to the desired skin layer (i.e. interrogate the desired skin layer) while the protruding member is in pressure contact with the skin of the patient under monitor. Also advantageously, in the case of a resilient protruding member where the pressure applied by the protruding member may be controlled, for example through the use of a resilient mechanism within the body of the device to resiliently bias the protruding member against the body of the device, the interrogation of specific skin layers is better controlled and can thus be adjusted according to the different skin types of different patients.

Numerous modifications could be made to the embodiments above without departing from the scope of the present invention.

1. A non-invasive probing device wearable over a skin region of a patient for physiological monitoring thereof, said probing device comprising:
a physiological probe having a sensing interface;
a housing for housing said probe, said housing comprising a body having a skin-side surface for lying adjacent to the skin region of the patient, said housing further comprising a fixed protruding member protruding from said skin-side surface away from said body and toward the skin region of the patient, said protruding member housing said sensing interface; and
attachment means for attaching said housing to the skin region of the patient and holding said skin-side surface and protruding member in pressure contact over the skin region.

2. A non-invasive probing device according to claim 1, wherein said sensing interface comprises at least one output of a light guide.

3. A non-invasive probing device according to claim 1, wherein said sensing interface comprises at least one input of a light guide.

4. A non-invasive probing device according to claim 1, wherein said attachment means comprise an adhesive layer extending over said skin-side surface of said body.

5. A non-invasive probing device according to claim 4, wherein said adhesive layer surrounds the protruding member and has a thickness less than a length of the protruding member.

6. A non-invasive probing device according to claim 1, wherein said attachment means comprise a flexible band or strap for encircling a body part of said patient and holding said housing of said probe in pressure contact over the skin region of the patient.

7. A non-invasive probing device according to claim 6, wherein said strap or said band is elastic.

8. A non-invasive probing device according to claim 1, wherein said attachment means comprise medical-grade adhesive tape for taping over said housing and securing said housing to the skin region of the patient, said medical-grade adhesive tape comprising adhesive strips for adhering to the housing and the skin region of the patient and thereby securing the housing to the skin region of the patient.

9. A non-invasive probing device according to claim 1, wherein said attachment means comprise a medical-grade adhesive patch comprising a non-adhesive portion for covering the housing and an adhesive portion for adhering to the skin region of the patient, thereby securing said housing to the skin region of the patient.

10. A non-invasive optical probing device according to claim 1, further comprising a rigid protective cover enclosing said housing, said cover having an outlet therein for the protruding member.

11. A non-invasive probing device wearable over a skin region of a patient for physiological monitoring thereof, said probing device comprising:
a physiological probe having a sensing interface;
a housing for housing said probe, said housing comprising a body having a skin-side surface for lying adjacent to the skin region of the patient, said housing further comprising a resilient protruding member resiliently protruding from said skin-side surface away from said body and toward the skin region of the patient, said resilient protruding member housing said sensing interface; and
attachment means for attaching said housing to the skin region of the patient and holding said skin-side surface and protruding member in pressure contact over the skin region.

12. A non-invasive probing device according to claim 11, wherein said sensing interface comprises at least one output of a light guide.

13. A non-invasive probing device according to claim 11, wherein said sensing interface comprises at least one input of a light guide.

14. A non-invasive probing device according to claim 11, wherein said resilient protruding member is retractable within said body.

15. A non-invasive probing device according to claim 11, wherein said resilient protruding member is extendable out of said body.

16. A non-invasive probing device according to claim 11, further comprising a resilient mechanism for resiliently biasing said resilient protruding member against said body.

17. A non-invasive probing device according to claim 16, wherein said resilient mechanism comprises a spring for resiliently biasing said resilient protruding member against a surface opposite said skin-side surface of said body.

18. A non-invasive probing device according to claim 17, further comprising a limit sensor operably connected between the spring and the body of the housing.

19. A non-invasive optical probing device according to claim 16, further comprising a rigid protective cover enclosing said housing, said cover having an outlet therein for the protruding member.

20. A non-invasive probing device according to claim 19, wherein said resilient mechanism comprises an elastic band for resiliently biasing said body against said protective cover.

21. A non-invasive probing device according to claim 11, wherein said attachment means comprise an adhesive layer extending over said skin-side surface of said body.

22. A non-invasive probing device according to claim 21, wherein said adhesive layer surrounds the protruding member and has a thickness less than a length of the resilient protruding member.

23. A non-invasive probing device according to claim 11, wherein said attachment means comprise a flexible band or strap for encircling a body part of said patient and holding said housing of said probe in pressure contact over the skin region of the patient.

24. A non-invasive probing device according to claim 11, wherein said attachment means comprise medical-grade adhesive tape for taping over said housing and securing said housing to the skin region of the patient, said medical-grade adhesive tape comprising adhesive strips for adhering to the housing and the skin region of the patient and thereby securing the housing to the skin region of the patient.

25. A non-invasive probing device according to claim 11, wherein said attachment means comprise a medical-grade adhesive patch comprising a non-adhesive portion for covering the housing and an adhesive portion for adhering to the skin region of the patient, thereby securing said housing to the skin region of the patient.
